(19) United States
(12) Patent Application Publication
(10) Pub. No.: US 2012/0157749 A1
(43) Pub. Date: Jun. 21, 2012

(54) HEAT THERAPY

(75) Inventors: Gwo-Bin Lee, Tainan City (TW); Xi-Zhang Lin, Tainan City (TW); Sheng-Chieh Huang, Tainan City (TW); Yi-Yuan Chang, Kaohsiung City (TW); Chiung-Yu Chen, Tainan City (TW); Yan-Shen Shan, Tainan City (TW)

(73) Assignee: INDUSTRIAL TECHNOLOGY RESEARCH INSTITUTE, Hsinchu (TW)

(21) Appl. No.: 12/968,293

(22) Filed: Dec. 15, 2010

(51) Int. Cl. A61N 2/00

(52) U.S. Cl. 600/10

(57) ABSTRACT

A heat therapy for thermally treating a target tissue within a living body by using a treating apparatus including a treating device with a magnetic part is provided. The magnetic part is heated to a first temperature by a high frequency electromagnetic field with a heating rate ranging from 1 to 5° C./sec, wherein the magnetic part is contacted the target tissue. Temperature controlling steps are performed to the magnetic part having the first temperature, and therefore the magnetic part has a final temperature higher than the first temperature, and a treatment is performed to the target tissue under the final temperature. Each of the temperature controlling steps is a heating step, a cooling step or a temperature conservation step, and the magnetic part is heated or cooled by the high frequency electromagnetic field with a heating rate or a cooling rate ranging from 1 to 5° C./sec.

### BACKGROUND OF THE DISCLOSURE

1. Field of the Invention

The disclosure relates to a heat therapy. More particularly, the disclosure relates to a heat therapy with a high frequency electromagnetic field.

2. Description of Related Art

The electromagnetic heat therapy is a newly developed treatment. In the electromagnetic heat therapy, the tissue is heated to cauterize, excise or stop bleeding by a treating device with a high temperature generated by an electromagnetic field. However, the treating device is prone to adhere to the tissue when the treating device is heated rapidly to a high temperature in a short time. Accordingly, the possible laceration or severe damage of the tissue may be caused when removing the treating device from the tissue.

Furthermore, a general heat therapy is merely performed to the tissue having a specific area. That is, the heat therapy has to be performed repeatedly to the tissue when the tissue has a large area, which delays the time for saving patients. Therefore, a new heat therapy is demanded to prevent the tissue from being adhered to the treating device and treat the tissue efficiently.

### SUMMARY OF THE INVENTION

A heat therapy is introduced herein, the heat therapy includes a plurality of the temperature controlling steps to prevent the magnetic part of the treating device from adhering to the target tissue, and the heat therapy is suitable for thermally treating the tissue with a large area.

A heat therapy for thermally treating a target tissue within a living body by using a treating apparatus is introduced herein, wherein the treating apparatus includes a treating device having a magnetic part. The heat therapy includes the following steps. The magnetic part is heated to a first temperature by a high frequency electromagnetic field with a heating rate ranging from 1° C./sec to 5° C./sec, wherein the magnetic part is contacted with the target tissue. A plurality of the temperature controlling steps is performed to the magnetic part having the first temperature, and therefore the magnetic part has a final temperature higher than the first temperature, wherein each of the temperature controlling steps is a heating step, a cooling step or a temperature conservation step, and the magnetic part is heated or cooled by the high frequency electromagnetic field with a heating rate or a cooling rate ranging from 1° C./sec to 5° C./sec. A treatment is performed to the target tissue under the final temperature.

In light of the foregoing, the heat therapy in the disclosure includes a plurality of temperature controlling steps, and therefore the magnetic part of the treating device is heated or cooled with an appropriate rate or the temperature of the magnetic part is kept constant. Accordingly, the magnetic part is prevented from adhering to the target tissue due to being rapidly heated in a short time, and an appropriate treatment can be performed to the target tissue in the temperature controlling step. Therefore, in the heat therapy of the disclosure, the tissue is prevented from adhering to the treating device, and thus, damage caused by removal of the treating device is not observed. Furthermore, the heat therapy is suitable for thermal treatment of the target tissue, such as hemostasis, cauterization or excision, and the large-area tissue can be thermally treated efficiently.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1 is a schematic diagram illustrating a treating apparatus according to the disclosure.

### DESCRIPTION OF EMBODIMENTS

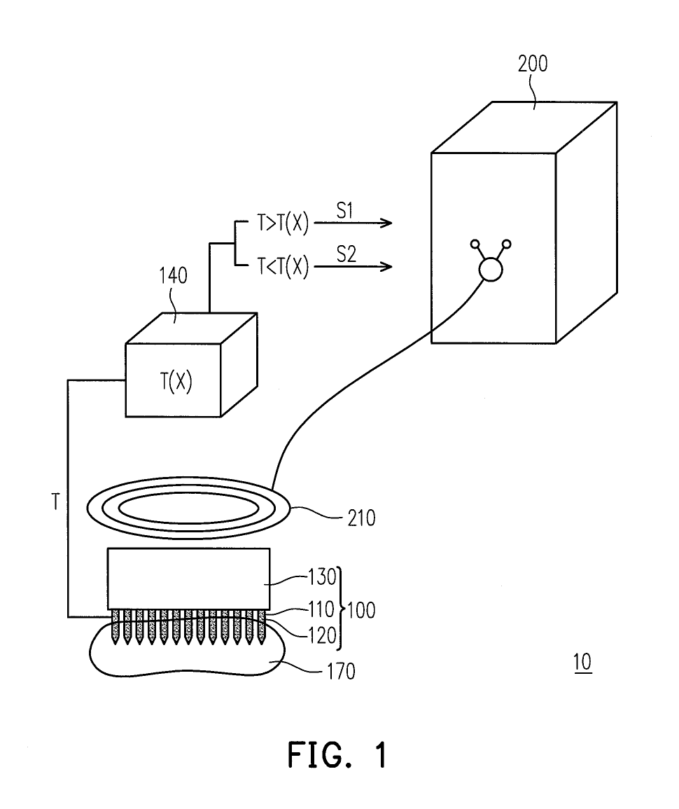

FIG. 1 is a schematic diagram illustrating a treating apparatus according to the disclosure. Referring to FIG. 1, the present exemplary embodiment provides a treating apparatus 10 includes a treating device 100, a temperature sensor 140 and a high frequency generating device 200. The treating device 100 is suitable to thermally treat the target tissue 170, and the treating device 100 includes the magnetic part 110 and the non-magnetic part 130. In the present exemplary embodiment, the treating device 100 is a needle assembly, for example. The non-magnetic part 130 can be a mount, and the magnetic part 110 can include a plurality of needles inserted to the mount, arranged in array and exposed externally. It should be illustrated that the treating device 100 can also be a needle with other configuration. Moreover, the treating device 100 can be a magnetic patch, a cutting blade, and so on. In other words, the magnetic part 110 can be a patch or a blade, and the non-magnetic part 130 can be a handheld part or a handle.

The magnetic part 110 is suitable for being heated to a temperature by high frequency electromagnetic field. The high frequency electromagnetic field is generated by the high frequency generating device 200. The magnetic part 110 is fabricated by a magnetic material, such as stainless steel and so on, that can generate heat under a magnetic field effect. In the present exemplary embodiment, the treating device 100 further includes an anti-adhesion layer 120. The anti-adhesion layer 120 is formed on a surface of the magnetic part 110, and the magnetic part 110 is in contact with the target tissue 170 through the anti-adhesion layer 120. The anti-adhesion layer 120 is configured to prevent the tissue and blood of the target tissue 170 from adhering to the magnetic part 110 due to high temperature. Thus, damage of the target tissue 170 caused by removal of the magnetic part 110 is not observed. The anti-adhesion layer 120 is fabricated by, for example, Teflon or ceramic material. Alternatively, by processing on the magnetic part 110, the anti-adhesion layer 120 is formed on the surface of the magnetic part 110, for example. However, in other exemplary embodiments, the magnetic part 110 may also have a surface without the anti-adhesion layer 120 formed thereon.

The non-magnetic part 130 is connected to the magnetic part 110. The non-magnetic part 130 is a part that is not affected by high frequency electromagnetic field, such that normal tissues or the operator are prevented from being damaged by high temperature. The non-magnetic part 130 is fabricated with a material that does not generate heat in a varying magnetic field. This material is a non-magnetic material such as ceramics, engineering plastic, heat resistant tape, and biocompatible gel. Herein, the engineering plastic is fabricated by, for example, polyetheretherkotone (PEEK), Teflon, and other materials. The heat resistant tape is fabricated with Teflon, for instance.

In the present exemplary embodiment, the high frequency generating device 200, for example, includes a coil 210. The coil 210 is driven by a current to generate a high frequency alternative electromagnetic field capable of heating the magnetic part 110. In the high frequency alternative electromagnetic field generated by the high frequency generating device 200, the magnetic part 110 generates an eddy current and hysteresis due to the high frequency alternative electromagnetic field. Consequently, the magnetic part 110 with a high temperature is used to cauterize or excise the target tissue 170 or stop the target tissue 170 from bleeding.

In the present exemplary embodiment, the temperature sensor 140 is connected to the magnetic part 110 and the high frequency generating device 200. The temperature sensor 140 measures a temperature T of the magnetic part 110 and outputs a sensing signal S1 or S2 to the high frequency generating device 200 according to the temperature T of the magnetic part 110. In the present exemplary embodiment, a part of the temperature sensor 140 can be fixed on the magnetic part 110 to measure the temperature T, and the temperature sensor 140 includes a thermocouple. In details, the temperature sensor 140 has a preset temperature T(X), and the sensing signal S1 indicates the high frequency generating device 200 to reduce the output power when the temperature T of the magnetic part 110 is higher than the preset temperature T(X). By contrast, when the temperature T of the magnetic part 110 is lower than the preset temperature T(X), the sensing signal S2 indicates the high frequency generating device 200 to increase the output power. In other words, the sensing signal S1 or S2 output by the temperature sensor 140 performs a feedback control to the high frequency generating device 200 according to the temperature T of the magnetic part 110. As a consequence, the magnetic part 110 is heated or cooled to the preset temperature T(X) by the high frequency electromagnetic field generated by the high frequency generating device 200, and thus, the target tissue 170 is thermally treated by the magnetic part 110 under the preset temperature T(X). It is noted that the preset temperature T(X) is substantially referred to T(X)±3° C., for example.

A heat therapy of the disclosure is illustrated with the treating apparatus of the FIG. 1, for example. The heat therapy is suitable for thermally treating a target tissue 170 within the living body by using a treating apparatus 10, wherein the treating apparatus 10 includes a treating device 100 having a magnetic part 110. The heat therapy includes the following steps. First, the magnetic part 110 is contacted with the target tissue 170, and the magnetic part 110 is heated to a first temperature T1 by a high frequency electromagnetic field with a heating rate ranging from 1° C./sec to 5° C./sec. Next, a plurality of the temperature controlling steps is performed to the magnetic part 110 having the first temperature T1, and therefore the magnetic part 110 has a final temperature Tf higher than the first temperature T1. Each of the temperature controlling steps is a heating step, a cooling step or a temperature conservation step, and the magnetic part 110 is heated or cooled with a heating rate or a cooling rate ranging from 1° C./sec to 5° C./sec. Then, a treatment is performed to the target tissue 170 under the final temperature Tf.

In the heat therapy of the disclosure, the process to change the temperature of the magnetic part 110 from the first temperature T1 to the final temperature Tf includes a plurality of the temperature controlling steps. The temperature controlling steps can include a plurality of heating steps, a plurality of cooling steps and a plurality of temperature conservation steps. In an exemplary embodiment, the temperature controlling steps can include performing a plurality of the heating steps and a plurality of the temperature conservation steps alternately. In another exemplary embodiment, the temperature controlling steps can include performing a plurality of the heating steps, a plurality of the cooling steps and a plurality of the temperature conservation steps performed in arbitrary order. Otherwise, in still another exemplary embodiment, the temperature controlling steps can include performing a plurality of the heating steps with different heating rates.

In the present exemplary embodiment, a frequency of the high frequency electromagnetic field is adjusted by controlling the output power of the high frequency generating device 200. Thus, the heating step, the cooling step or the temperature conservation step is performed. It is noted that the output power of the high frequency generating device 200 is reduced to about 8% in the temperature conservation step rather than be shut down, and the temperature of the magnetic part 110 in the temperature conservation step is hardly influenced by the output power. Therefore, the temperature of the magnetic part 110 is kept constant.

A design of the heat therapy is illustrated according to an exemplary embodiment of the disclosure. In the present exemplary embodiment, the heat therapy includes performing a plurality of the heating steps and a plurality of the temperature conservation steps alternately. Referring to FIG. 1, in the present exemplary embodiment, the magnetic part 110 with needles is inserted into the target tissue 170. The temperature sensor 140 is connected to the magnetic part 110 and the high frequency generating device 200. Thus, as mentioned above, a sensing signal S1 or S2 generated by the temperature sensor 140 according to the temperature T of the magnetic part 110 may be transmitted to the high frequency generating device 200. Herein, the temperature of the target tissue 170 is about 36° C., and room temperature is about 28° C.

Then, the magnetic part 110 is heated to a first temperature T1 by a high frequency electromagnetic field, wherein the high frequency electromagnetic field is generated by the high frequency generating device 200. The magnetic part 110 is heated with a heating rate ranging from 1° C./sec to 5° C./sec. In the present exemplary embodiment, the first temperature T1 is 50° C., for example. As mentioned above, the sensing signal S1 or S2 output by the temperature sensor 140 performs a feedback control to the high frequency generating device 200. In the present exemplary embodiment, a preset temperature T(X) of the temperature sensor 140 is 50° C., and thus the magnetic part 110 is heated to the preset temperature T(X) by the high frequency electromagnetic field.

Next, a temperature conservation step is performed to the magnetic part 110. Thus, the magnetic part 110 is kept at the first temperature T1. In this step, output power of the high frequency generating device 200 can be adjusted to about 8%, and a time for the temperature conservation step is about 10 seconds.

Thereafter, the magnetic part 110 is heated to a second temperature T2 by the high frequency electromagnetic field, wherein the high frequency electromagnetic field is generated by the high frequency generating device 200. The magnetic part 110 is heated with a heating rate ranging from 1° C./sec to 5° C./sec. In the present exemplary embodiment, the second temperature T2 is 80° C., for example. Accordingly, a preset temperature T(X) of the temperature sensor 140 is 80° C., for example.

Afterwards, a temperature conservation step is performed to the magnetic part 110. Thus, the magnetic part 110 is kept at the second temperature T2. In this step, output power of the high frequency generating device 200 can be adjusted to about 8%, and a time for the temperature conservation step is about 10 seconds.

Then, the magnetic part 110 is heated to a third temperature T3 by the high frequency electromagnetic field, wherein the high frequency electromagnetic field is generated by the high frequency generating device 200. The magnetic part 110 is heated with a heating rate ranging from 1° C./sec to 5° C./sec. In the present exemplary embodiment, the third temperature T3 is 100° C., for example. Accordingly, a preset temperature T(X) of the temperature sensor 140 is 100° C., for example.

Next, a temperature conservation step is performed to the magnetic part 110. Thus, the magnetic part 110 is kept at the third temperature T3. In this step, output power of the high frequency generating device 200 can be adjusted to about 8%, and a time for the temperature conservation step is about 10 seconds.

Thereafter, the magnetic part 110 is heated to a final temperature Tf by the high frequency electromagnetic field, wherein the high frequency electromagnetic field is generated by the high frequency generating device 200. The magnetic part 110 is heated with a heating rate ranging from 1° C./sec to 5° C./sec. In the present exemplary embodiment, the final temperature Tf is 150° C., for example. Accordingly, a preset temperature T(X) of the temperature sensor 140 is 150° C., for example.

Afterwards, a temperature conservation step is performed to the magnetic part 110. Thus, the magnetic part 110 is kept at the final temperature Tf. In this step, output power of the high frequency generating device 200 can be adjusted to about 8%, and a time for the temperature conservation step is about 10 seconds.

In the temperature conservation step, a treatment is performed to the target tissue 170 under the final temperature Tf. The treatment can include hemostasis, cauterization and excision. In the present exemplary embodiment, the treatment including excision and hemostasis is performed to the target tissue 170. In other embodiment, the final temperature Tf is adjusted to about 100° C. when cauterization is performed to the target tissue 170. In other embodiment, the final temperature Tf is adjusted to about 150° C. when cauterization and hemostasis are performed to the target tissue 170 at the same time. After performing the treatment, the heat therapy is finished. In the present exemplary embodiment, a total time of the heat therapy is about 5 minutes, for example. In other embodiment, the final temperature ranges from 100° C. to 150° C.

In the present exemplary embodiment, the heat therapy is performed with a plurality of the temperature controlling step, and the magnetic part 110 is heated with a heating rate ranging from 1° C./sec to 5° C./sec. Therefore, the magnetic part 110 of the treating device 100 is prevented from adhering to the target tissue 170 due to being rapidly heated in a short time, and the possible laceration or severe damage of the target tissue 170 caused by removal of the magnetic part 110 is not observed. Furthermore, the magnetic part 110 may have a good conductivity because the target tissue 170 is not adhered to the magnetic part 110. Accordingly, the heat therapy is suitable for thermal treatment of the target tissue 170, such as hemostasis, cauterization or excision, and the large-area tissue can be thermally treated efficiently.

It is noted that the heat therapy of the present exemplary embodiment includes performing a plurality of the heating steps and a plurality of the temperature conservation steps alternately, and the invention is not limited thereto. For example, the heat therapy may further include the cooling steps. The treatment is performed at low temperature, such as medicament injection. In other words, medicament injection to the target tissue is performed in the temperature conservation step with a low temperature and after the cooling step. In addition, if required, the treatments, such as hemostasis, cauterization and excision, may be performed in any one of the heating steps, the temperature conservation steps, and the cooling steps. Generally, the heat therapy is designed based on the treatments needed to be performed to the target tissue. Therefore, the treatments can be performed at appropriate temperature and time selected by medical staff.

Herein, an experiment data is provided. In the experiment, the heat therapy according to an exemplary embodiment of the disclosure and a conventional heat therapy are performed to porcine livers respectively by using the treating apparatus 10 illustrated in FIG. 1. In detail, the heat therapy according to the exemplary embodiment of the disclosure includes the following steps. The magnetic part 110 is heated to 60° C. from room temperature and kept at 60° C. for 10 seconds, heated to 80° C. from 60° C. and kept at 80° C. for 10 seconds, heated to 100° C. from 80° C. and kept at 100° C. for 10 seconds, and heated to 120° C. from 100° C. and kept at 120° C. for 5 minutes. In the heat therapy according to the exemplary embodiment of the disclosure, a heating rate in each heating step is 1° C./sec. In contrast, in the conventional heat therapy, a porcine liver is directly heated to 120° C. from room temperature and kept at 120° C. for 5 minutes, wherein a heating rate is 1° C./sec. The treating devices are removed from the porcine livers after the heat therapy is finished, and cauterization of the porcine livers and the magnetic part of the treating devices are observed respectively. In the porcine liver treated with the conventional heat therapy, burned black tissue is observed in the cauterizing region, and area of the cauterizing region is 297 mm²(33 mm×9 mm). Furthermore, tissue is adhered to a surface of the magnetic part. On contrary, in the porcine liver treated with the heat therapy according to the exemplary embodiment of the disclosure, burned black tissue is not observed in the cauterizing region, and area of the cauterizing region is 468 mm²(36 mm×13 mm). Moreover, tissue is not adhered to a surface of the magnetic part. In other words, by the heat therapy of the disclosure, the magnetic part of the treating device is prevented from adhering to the target tissue when being heated. Thus, the target tissue is not burned black, and the magnetic part of the treating device may have a better conductivity. Accordingly, compared with the conventional heat therapy, the heat therapy of the disclosure is suitable for cauterizing a large-area tissue and has good curative effect on the target tissue.

In summary, the heat therapy in the disclosure includes a plurality of temperature controlling steps, and each of the temperature controlling steps is a heating step, a cooling step or a temperature conservation step. The magnetic part is heated or cooled by the high frequency electromagnetic field with a heating rate or a cooling rate ranging from 1° C./sec to 5° C./sec or the temperature of the magnetic part is kept constant. In other words, the magnetic part is not rapidly heated in a short time, and thus the magnetic part is prevented from adhering to the target tissue. Therefore, in the heat therapy in the disclosure, the tissue is prevented from the damage caused by removal of the treating device. Furthermore, the heat therapy is designed based on the treatments to be performed to the target tissue. Therefore, the treatments, such as hemostasis, cauterization or excision, can be performed at appropriate temperature and time, and have good curative effect on the target tissue.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A heat therapy for thermally treating a target tissue within a living body by using a treating apparatus, wherein the treating apparatus includes a treating device having a magnetic part, the steps of the heat therapy comprising:
heating the magnetic part to a first temperature by a high frequency electromagnetic field with a heating rate ranging from 1° C./sec to 5° C./sec, wherein the magnetic part is contacted with the target tissue;
performing a plurality of temperature controlling steps to the magnetic part having the first temperature, and therefore the magnetic part having a final temperature higher than the first temperature, wherein each of the temperature controlling steps is a heating step, a cooling step or a temperature conservation step, and the magnetic part is heated or cooled by the high frequency electromagnetic field with a heating rate or a cooling rate ranging from 1° C./sec to 5° C./sec; and
performing a treatment to the target tissue under the final temperature.

2. The heat therapy as claimed in claim 1, wherein the treating device comprises a high frequency generating device, and the steps of the heat therapy further comprising:
generating the high frequency electromagnetic field by the high frequency generating device.

3. The heat therapy as claimed in claim 2, wherein a frequency of the high frequency electromagnetic field is adjusted by controlling an output power of the high frequency generating device to perform the heating step, the cooling step or the temperature conservation step.

4. The heat therapy as claimed in claim 3, wherein in the temperature conservation step, the output power of the high frequency generating device can be adjusted to about 8%.

5. The heat therapy as claimed in claim 3, wherein the treating device comprises a temperature sensor, the temperature sensor is connected to the magnetic part and the high frequency generating device, and the steps of the heat therapy further comprising: measuring a temperature of the magnetic part and outputting a sensing signal to the high frequency generating device according to the temperature of the magnetic part.

6. The heat therapy as claimed in claim 5, wherein the temperature sensor has a preset temperature, the output power of the high frequency generating device is reduced according to the sensing signal when the temperature of the magnetic part is higher than the preset temperature, and the output power of the high frequency generating device is increased according to the sensing signal when the temperature of the magnetic part is lower than the preset temperature.

7. The heat therapy as claimed in claim 5, wherein the temperature sensor comprises a thermocouple.

8. The heat therapy as claimed in claim 1, wherein a plurality of the temperature controlling steps comprises performing a plurality of the heating steps and a plurality of the temperature conservation steps alternately.

9. The heat therapy as claimed in claim 1, wherein medicament injection to the target tissue is performed in the temperature conservation step with a low temperature and after the cooling step.

10. The heat therapy as claimed in claim 1, wherein the final temperature ranges from 100° C. to 150° C.

11. The heat therapy as claimed in claim 1, wherein the treatment comprises hemostasis, cauterization or excision.

12. The heat therapy as claimed in claim 1, wherein the magnetic part comprises a needle, a magnetic patch or a cutting blade.

13. The heat therapy as claimed in claim 1, wherein an anti-adhesion layer is formed on a surface of the magnetic part, and the magnetic part is in contact with the target tissue through the anti-adhesion layer.

14. The heat therapy as claimed in claim 13, wherein the anti-adhesion layer comprises Teflon or ceramic material.

15. The heat therapy as claimed in claim 1, wherein a total time of the heat therapy is about 5 minutes.
